(19) United States
(12) Patent Application Publication
(10) Pub. No.: US 2012/0157802 A1
(43) Pub. Date: Jun. 21, 2012

(54) ELECTROCARDIOGRAPHIC DATA ACQUISITION DEVICE

(76) Inventor: Chang-An Chou, Taipei (TW)

(21) Appl. No.: 13/392,099

(22) PCT Filed: Aug. 24, 2010
(86) PCT No.: PCT/CN2010/001285

§ 371 Date: Feb. 24, 2012

(30) Foreign Application Priority Data
Aug. 24, 2009 (CN) 200910163088.8

(51) Int. Cl. A61B 5/0205; A61B 5/0404

(52) U.S. Cl. 600/324; 600/523

(57) ABSTRACT

An ECG data acquisition device (10) can be mechanically engaged with a portable device (20) to form a combined unit. The ECG data acquisition device (10) includes a housing (11), at least two electrodes located on the surface of the housing (11) for obtaining ECG signals from a user, and a circuit assembly (100). The circuit assembly (100) includes an analog signal processing module (102), a processing module (104) having an analog/digital converter (1041), and a processor (1042) to execute a preloaded program to perform the ECG acquisition and data processing, and a communication module (106) for communicating with the portable device (20).

### FIELD OF THE INVENTION

The present invention is related to an electrocardiographic data acquisition device, and more particularly to a device which can be integrated with a portable device to form a combined unit for performing a handheld ECG data acquisition.

### BACKGROUND OF THE INVENTION

Many portable devices for ECG examination are developed for satisfying the growing demands of professional medical personnel and patients. For example, Digital Walk FM-150, by Fukuda Denshi, and DR200/HE, by NorthEast Monitoring, provide ECG examinations via lead-wire electrodes attached to the user, and InstantCheck, by DailyCare Biomedical, allows the user to perform ECG examination via the electrodes on the housing.

However, to carry such a device is an extra weight and volume for the user, especially, the user may already own many portable electronic devices, such as, mobile phone, MP3 player, and PDA. Therefore, just as the trend to equip the electronic device multiple functions for simplification, such as, the mobile phone now usually can function as a phone, a camera, a notepad, and a music player, if the ECG examining device also can be integrated with the portable device, it is believed that the convenience and portability for the ECG examining device can have a significant improvement.

U.S. Pat. No. 6,546,232 and U.S. Pat. No. 7,433,718 provided the possibility for the mobile phone to directly have the electrodes mounted thereon. However, this also means that the user has to make a compromise as selecting the mobile phone. Besides, since the electrodes and the phone are inseparable, the electrodes can not be removed when the physiological examination is no longer needed, and oppositely, the function of physiological examination might also become ineffective as the mobile phone becomes malfunction.

Consequently, there is the need to develop an ECG examining device which can be achieved by integrating with a common portable device, so that the user can possess the function of ECG examination without sacrificing the selection to the portable device.

The object of the present invention is to provide an ECG data acquisition device which can be engaged with a common portable device to form a combined unit, so as to increase the possibility for being carried and used by the user.

Another object of the present invention is to provide an ECG data acquisition device integrated with a portable device, so that the user can conveniently perform the ECG data acquisition via a handheld manner.

A further object of the present invention is to provide an ECG data acquisition device which can utilize the hardware of a portable device to achieve the ECG examination, so as to reduce the volume and weight thereof, and also, the manufacturing cost.

### SUMMARY OF THE INVENTION

In one aspect of the present invention, an ECG data acquisition device which can be engaged with a portable device is provided. The ECG data acquisition device includes a housing, at least two electrodes located on the surface of the housing for obtaining ECG signals from a user, and a circuit assembly, wherein the circuit assembly includes an analog signal processing module, a processing module having an analog/digital converter, and a processor to execute a preloaded program to perform the ECG data acquisition and data processing, and a communication module, for communicating with the portable device. Here, the ECG data acquisition device, through the housing thereof, can be mechanically engaged with the portable device to form a combined unit. Then, to obtain the ECG signals, the user contacts at least one of the electrodes while holding the combined unit, and the other electrode is contacted to an appropriate position of the user. Furthermore, the processor executes the preloaded program to acquire the ECG data, and then, the ECG data is transmitted to the portable device.

Here, the portable device can be, but not limited, a mobile phone, a PDA, a MP3 Player, a GPS receiver, a portable/handheld game machine, a digital camera, or a digital video camera/recorder, that is, a portable device commonly used in the daily life, so that the possibility for the user to utilize the function of ECG examination can be increased.

When the ECG data acquisition device is integrated with the portable device, the communication therebetween can be implemented to be wired (such as, USB, UART, Ethernet, and 1394) or wireless (such as, Bluetooth, wireless USB, WiFi, and IrDa), without limitation. Preferably, it can be implemented that the ECG data is transmitted to the portable device for real-time processing and/or displaying and storage.

In a preferred embodiment, the processor, the analog processing module and/Or the analog/digital converter are implemented to be a sub-unit which can be removed from the housing for connecting with other electrodes so as to perform another ECG data acquisition.

Further, for adapting to the size variation of different portable devices, the ECG data acquisition device according to the present invention also can be implemented to have a mechanical structure adjustable to match each portable device. On the other hand, it also can be implemented to provide plural housings which respectively can adapt to different kinds of portable devices, for example, housings respective for mobile phone, PDA, and MP3 Player, etc.

The electrodes on the surface of the housing also provide the convenience for the user to perform the handheld operation, so that as one of the user's hands holds the combined unit, at least one of the electrodes can contact therewith. Here, the quantity, position and type of the electrodes can be varied in accordance with different requirements. For example, in a preferred embodiment, the electrodes can be implemented to be removable for exchanging for another type of electrodes, e.g., from dry electrodes to wet gel electrodes; and in another preferred embodiment, plural housings which respectively have different arrangements of electrodes can be provided, so that through changing the housing, the device can provide the user different hand-holding modes to perform the ECG data acquisition.

Furthermore, the ECG data acquisition device of the present invention also can have an extension port for connecting at least an electrode and/or sensor, such as, via a wire, so as to provide the user the opportunity to expand the function. For example, the electrode(s) can be connected for providing a more advanced capability of ECG data acquisition, e.g., from 2-lead to 12-lead, that is, the connected electrode(s) is implemented to be the additional electrode(s) for cooperating with the electrodes on the housing; or the electrodes connected via the extension port also can be implemented to replace the original electrodes, and in this case, the combined unit may be operated similar to a Holter. On the other hand, a sensor also can be connected, for example, an oximeter can be provided to cooperate with the ECG examination, so as to achieve a cross reference therebetween for increasing data accuracy. In another preferred embodiment, the oximeter also can be included in the ECG data acquisition device for providing blood physiological parameters in each ECG examination. Hence, there is no limitation.

In another aspect of the present invention, the ECG data acquisition device according to the present invention also can be implemented to use an electrical connection interface to connect with the portable device, and the processor of the portable device is utilized to execute a preloaded program to obtain the ECG data through the electrodes on the housing of the ECG data acquisition device, and then process and display the ECG data.

Here, the analog signal processing module and/or the analog/digital converter which are needed for performing physiological signal acquisition can be included in the ECG signal acquisition device and/or the portable device, without limitation. In particular, when the ECG signal acquisition device only provides the electrodes, it becomes a pure electrode structure which can be mechanically engaged with the portable device to facilitate the handheld operation.

Consequently, by providing the ECG data/signal acquisition device which can be mechanically integrated with the portable device, the present invention can furnish an extra function for a common portable device. Hence, the user can conveniently obtain the information about the heart while carrying the most frequently used portable device (e.g., the mobile phone, PDA, or MP3 Player) in the daily life, that is, the user does not need to carry another device for performing the ECG examination. Then, since the ECG data/signal acquisition device and the portable device can be integrated together to form a combined unit, the electrodes mounted on the surface of the housing can provide a great contribution to the handheld operation. Moreover, through the cooperation with the portable device, the manufacturing cost of the ECG data/signal acquisition device can accordingly be reduced.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1A is a schematic view showing the assembling of an ECG data acquisition device and a portable device according to a preferred embodiment of the present invention;

FIG. 1B is a rear view illustrating a first example of the combined unit in FIG. 1A;

FIG. 1C is a top view showing the user holds the combined unit of FIG. 1A to perform the ECG data acquisition;

FIG. 1D is a rear view illustrating a second example of the combined unit in FIG. 1A;

FIGS. 2A-2G are schematic views illustrating the combination examples of the ECG data acquisition device and the portable device according to the present invention;

FIG. 3 is a schematic view showing the circuitry of the ECG data acquisition device according to a preferred embodiment of the present invention;

FIG. 4A is a schematic view illustrating the ECG data acquisition device is connected with additional electrodes according to a preferred embodiment of the present invention; and

FIG. 4B is a schematic view illustrating the ECG data acquisition device is connected with a sensor according to another preferred embodiment of the present invention.

### DETAILED DESCRIPTION OF THE PREFERRED EMBODIMENTS

Generally, for all different kinds of purposes, people may carry one or more portable devices everyday, including, but not limited, mobile phone, MP3 Player, PDA, GPS receiver, digital camera, digital video camera/recorder, and/or portable/handheld game machine. And, the concept of the present invention is to provide a device which can equip the common portable device an additional function of ECG examination through a mechanical combination, so as to offer a more economical and less space-occupied manner for the user to possess the ECG examining device and thus increase the probability of the device to be utilized.

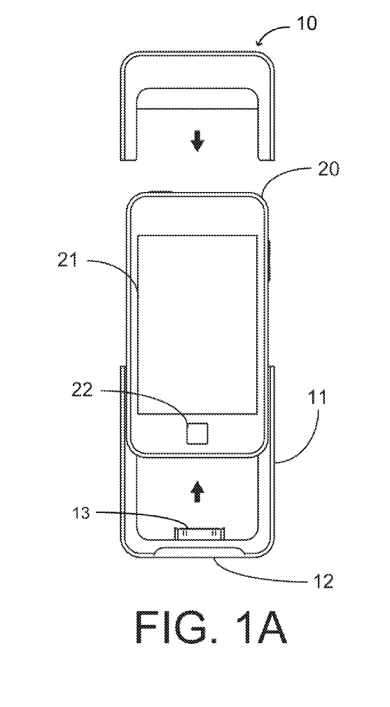

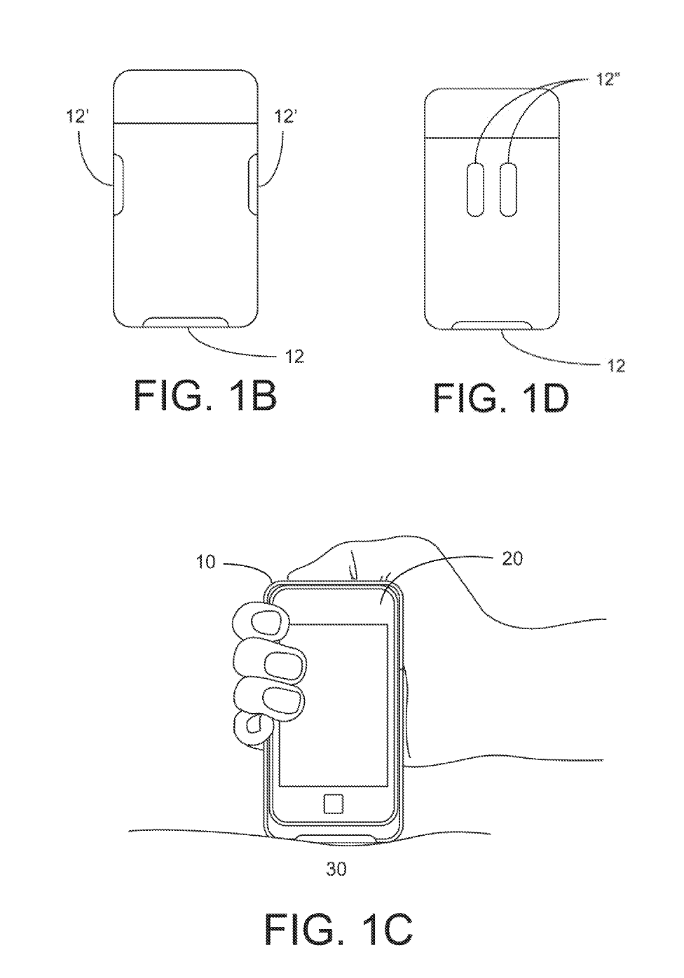

Now, please refer to FIG. 1A which is a schematic view showing the assembling of an ECG data acquisition device and a portable device in a preferred embodiment of the present invention, and FIG. 1B which is a rear view showing a combined unit of FIG. 1A. As shown, the ECG data acquisition device 10 which can be engaged with the portable device 20 includes a housing 11 and three electrodes 12, 12′ mounted on the surfaces of the housing 11. Particularly, the housing 11 is implemented to be able to mechanically integrate with the portable device 20 to form a combined unit, and through the cooperation therebetween, the user can directly operate the combined unit to acquire ECG data in a handheld manner. For example, as shown in FIG. 1C, when the user's right hand holds the right and left sides of the combined unit, at the same time, the user's hand will contact two electrodes 12′ respectively mounted thereon, and then, after the user holds the combined unit to further contact the lower electrode 12 with another body position 30, such as, the limb (e.g., left hand) or the chest, the ECG data acquisition can be achieved. Alternatively, as shown in FIG. 1D, it also can be implemented to have the electrodes 12″ mounted on the rear side of the combined unit for contacting with the holding hand of the user.

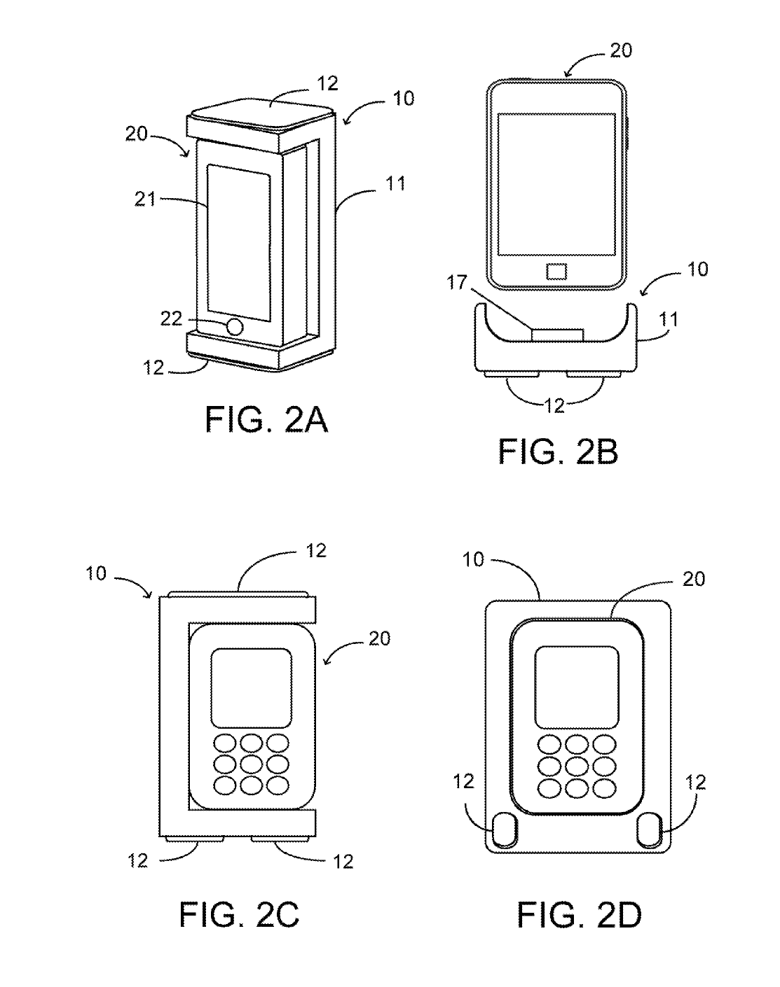

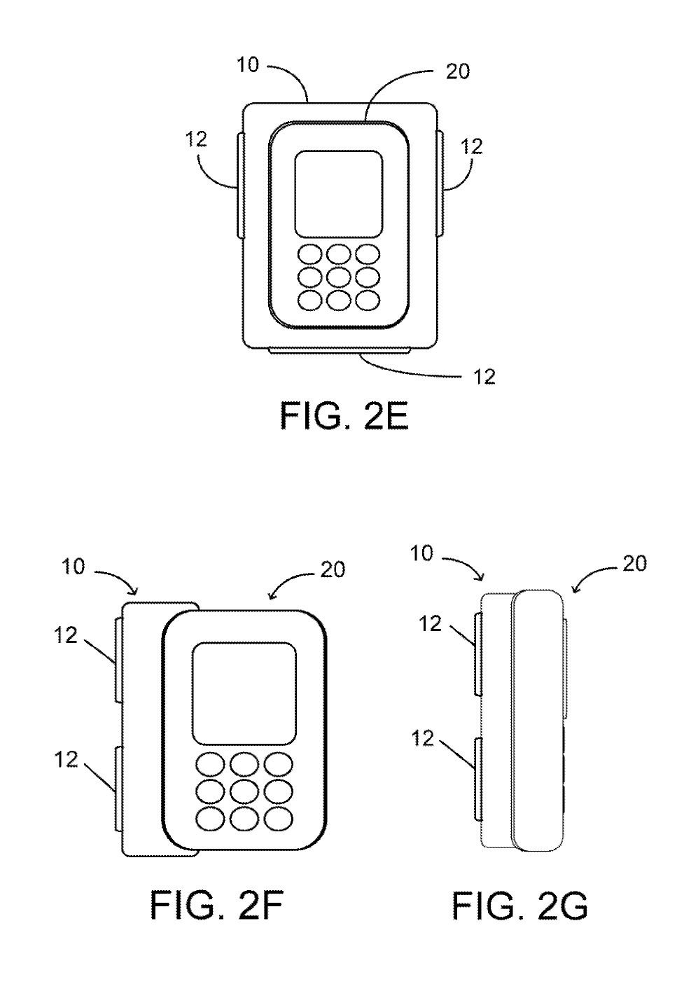

There are various ways can be used to achieve the combination between the ECG data acquisition device and the portable device. For example, the combination can be achieved by magnetic force, adhesives, the material flexibility of housings, and/or corresponding physical structures of two housings, such as, a slideway (FIG. 1A), a slot, a buckle, and a fastener, or the connection of a connector (e.g., an electrical connector 13 in FIG. 1A or a USB connector 17 in FIG. 2B). That is, the point is to provide a secure combination between the two, and the achieving method is not limited.

As to the shape of the housing 11, it also can be varied as needed. For example, the ECG data acquisition device 10 can be formed to hold three sides of the portable device 20 (e.g., the top, the bottom and the rear sides (FIG. 2A), the left, the right and the bottom sides (FIG. 2B), and the top, the bottom and the left sides (FIG. 2C)), to hold the whole device 10 (FIG. 1A, FIG. 2D and FIG. 2E), or to hold one side only (FIG. 2F and FIG. 2G), so that there is no limitation to the shape of the housing 11. The point is that after combination, the combined unit is suitable for a handheld operation, and the electrodes are located at the positions that can contact with the user's holding hand and another body position. Here, since the portable device is always formed to have a volume and a shape suitable for the handheld operation, the combined unit formed according to the concept of the present invention also can provide a proper volume and shape for carrying and handheld operation.

Furthermore, for adapting to different portable devices 20 which might be varied in size, the housing 11 of the ECG data acquisition device 10 according to the present invention also can be implemented to have a mechanical structure which can be adjusted to match to size differences, for example, to adjust the length and the width, so as to provide a maximum adaptability.

Besides, it also should be noted that the quantity, the type and the position of the electrodes 12 also can be changed in accordance with different demands. For example, a reference electrode and/or ground electrode can be increased to improve accuracy. The quantity and position can be changed to conform to different kinds of ECG data acquisitions, e.g., in one embodiment, a set of housings with different electrode quantities/positions can be provided to the user to select a proper one. Moreover, the electrodes can be designed to be removable so as to benefit the replacement, such as, to exchange from a dry electrode to a wet gel electrode, or to alter the position arrangement of the electrodes. Particularly, a retractable structure can be provided to the electrode(s), so that when the electrode is contacted with the skin and suffered from the pressure, the structure may shrink to emphasis the contact, and/or a sensing element can be connected to the electrode(s) to sense the pressure applied thereon, so as to notify the user if the operation is proper. Besides, in addition to the electrodes for ECG data acquisition, the device also can be implemented to include a sensor for further completing the function, for example, an oximeter can provide blood physiological parameters for achieving a cross reference with the ECG data.

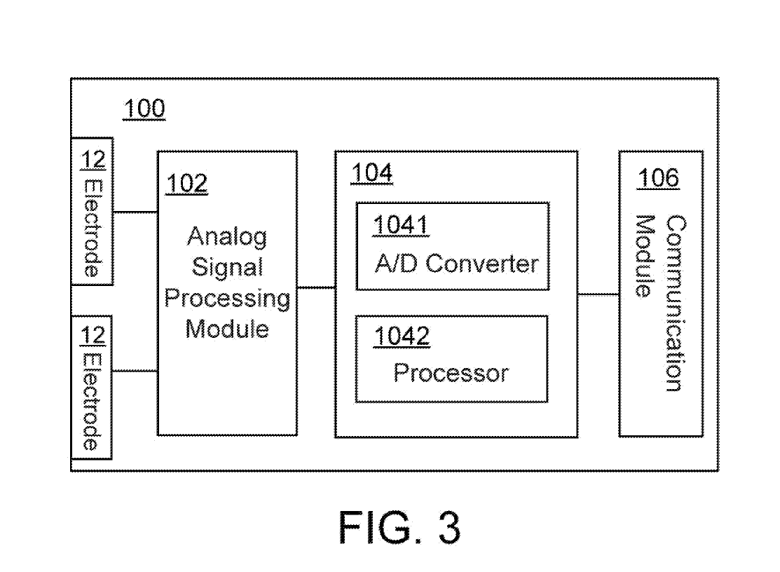

Then, please refer to FIG. 3, which is a schematic view showing the circuitry of the ECG data acquisition device according to a preferred embodiment of the present invention. In this embodiment, the circuit assembly 100 of the ECG data acquisition device 10 includes an analog signal processing module 102 connected to the electrodes 12 for processing the obtained ECG signals; a processing module 104 including an A/D converter 1041 for digitizing the analog data, and a processor 1042 for controlling the ECG data acquisition and processing the data; and a communication module 106 for communicating with the portable device 20. First, the analog signal processing module 102 can process the ECG signals, such as, amplifying and filtering. Then, the data is converted into digital data by the A/D converter 1041 and then transmitted to the processor 1042. Here, the processor 1042 is implemented to be a control center and to execute a preloaded program for performing the ECG data acquisition and for processing, calculating, and/or analyzing the data, so that the device 10 can further include a memory for storage and/or if appropriate, an analysis result can be produced. Then, through the communication module 106, the ECG data (and the analysis result) can be transmitted to the portable device 20 for further processing (such as, advanced calculation, analysis, and/or diagnosis) and/or being shown on the display. That is, the ECG data acquisition device 10 possesses the processor 1042 to control and perform the ECG data acquisition, and through the communication with the portable device 20, the operation interface of the portable device 20, such as, the display 21 and the keyboard 22, can be utilized to complete the ECG examination.

It should be noted, in addition to be included in the housing 11, the analog signal processing module, the A/D converter and/or the processor also can be positioned outside the housing 11 and connected via a connecting wire. Particularly, the analog signal processing module, the A/D converter and/or the processor can be implemented to form a sub-unit which can be removed from the housing to connect with other electrodes, so as to perform another ECG data acquisition, thereby the cost to possess multiple ECG examining devices can be further reduced. Hence, the arrangement of the circuitry assembly 100 can be varied according to the real situation, without limitation.

Here, the communication between the ECG data acquisition device 10 and the portable device 20 is controlled through the respective processors thereof. This communication can be wired or wireless. If the wired communication (e.g., USB, UART, Ethernet, 1394 or other wired communication modules) is employed, it will be necessary to simultaneously achieve an electrical connection therebetween as the two are integrated together. If it is implemented to utilize the wireless communication (e.g., Bluetooth, wireless USB, WiFi, IrDa, or other wireless communication modules), then the electrical connection between the two can be omitted. Notably, the ECG data can be transmitted to the portable device in real time, or the data can be stored and then transmitted after the acquisition process, without limitation.

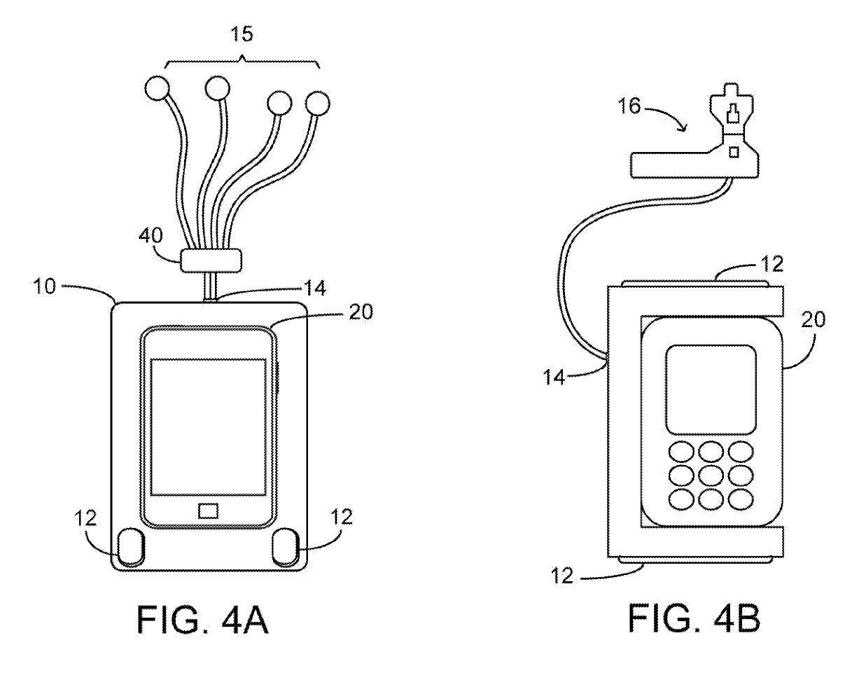

Advantageously, the ECG data acquisition device also can include a connecting port 14 for further connecting to at least an electrode/sensor 15. For example, the quantity of electrodes can be increased to achieve a more advanced ECG data acquisition (as shown in FIG. 4A), such as, from 2-lead to 12-lead. Here, the connected electrodes can be implemented to cooperate with the original electrode, or to replace them, and if the latter situation is employed, the device can be operated in a non-handheld manner, for example, through the lead-wire connected electrodes, the user can have the electrodes attached to the body but the device carried by a belt, just like a Holter. Therefore, the function of the ECG data acquisition device can accordingly be upgraded. On the other hand, additional sensor(s) also can be connected for expanding the function of the device, e.g., an oximeter (as shown in FIG. 4B) can be employed to provide blood physiological parameters so as to achieve a cross reference with ECG data. Particularly, as shown in FIG. 4A, a junction box 40 can be used to maximize the expansion possibility. Here, as the additional electrode(s)/sensor(s) are employed, the user only needs to download and execute corresponding program(s), and then, the device can be operated to have a different operation mode.

Preferably, the portable device can be implemented to connect to a network, e.g., Internet, in a wired or wireless manner, so that the acquired data (and the analysis result) can be transmitted to a medical personnel, a remote server, and/or a website, for obtaining more advanced calculation, analysis and/or diagnosis. Further, the programs for expanding the function, as described above, also can be downloaded through the network connection. For example, the remote server and/or the website can provide the programs of different kinds of ECG data acquisitions for the user to download, such as, for 2-lead mode, for 12-lead mode, for Holter mode, and for ECG plus oximeter mode. Accordingly, through downloading the program and rearranging the positions of electrodes by exchanging the housing or connecting with external electrodes, as mentioned above, the ECG data acquisition device according to the present invention can provide the functions of multiple kinds of ECG data acquisitions, so that not only the occupied space but also the cost can be saved.

As to the power supply to the ECG data acquisition device, it can be powered by its own power source, e.g., a battery, or an external power source, such as, the portable device, without limitation.

In another aspect of the present invention, the ECG data acquisition device 10 also can be implemented to not include the processor, that is, the ECG data acquisition is controlled and the data is processed by the processor in the portable device 20, so as to further reducing the manufacturing cost. Here, for achieving this, the ECG data acquisition device should be integrated electrically and mechanically with the portable device, so that the data can be obtained via the electrically connected electrodes under the control of the portable device's processor. Then, the corresponding program(s) for performing the ECG data acquisition should be downloaded to the portable device, e.g., from the remote server or the website. As to the analog signal processing module and the A/D converter which are necessary for physiological signal acquisition, they can be selected to respectively install in the ECG data acquisition device and/or the portable device or to locate outside the two (e.g., connected through the connecting wire(s)).

For example, in a particular embodiment, as the portable device 20 originally possesses the capability of processing analog signals (which means the analog signal processing module and the A/D converter are both installed therein), such as, a special made mobile phone, then the ECG data acquisition device can be implemented to be a pure electrode structure capable of mechanically and electrically integrating with the portable device for achieving the ECG examination, and in this case, a minimized volume of ECG data acquisition device can be obtained. Alternatively, in another particular embodiment, the analog signal processing module and the A/D converter also can be formed to be a unit to connect with the portable device 20 or the ECG data acquisition device 10, so that the user can carry the portable device 20 with the electrode structure integrated, and then, connect with the unit for processing analog signals as performing the ECG data acquisition. Therefore, there is no restriction on the implementation of the ECG data acquisition device, which only has to be able to obtain ECG signal/data via electrodes thereon from the user. As to other electronic components, they can be varied and modified in accordance with the real demands as performing the ECG data acquisition.

In the aforesaid, the present invention provides an ECG data acquisition device having a housing with electrodes mounted on the surface thereof for integrating with a common portable device, so as to offer the user a handheld manner to perform the ECG examination. Accordingly, without additionally carrying an ECG examining device, the portable device can obtain the ECG data acquisition function by slightly increasing the volume thereof through integrating with the ECG data acquisition device according to the present invention. Besides, through employing different kinds of ECG data acquisition devices (of different positions of electrodes) or connecting to external electrode(s)/sensor(s) as well as cooperating with a proper (downloaded) program, the user even can perform multiple kinds of ECG data acquisitions via the same portable device. Therefore, not only the occupied volume, but also the cost can be saved.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. An electrocardiographic data acquisition device for being engaged with a portable device, comprising:
a housing;
at least two electrodes, located on the surface of the housing for obtaining ECG signals from a user; and
a circuit assembly, comprising:
an analog signal processing module;
a processing module, comprising:
an analog/digital converter; and
a processor, to execute a preloaded program to perform an ECG data acquisition and data processing; and
a communication module, for communicating with the portable device, wherein
the ECG data acquisition device, through the housing thereof, is mechanically integrated with the portable device to form a combined unit;
to obtain the ECG signals, the user contacts at least one of the electrodes while holding the combined unit, and the other electrode is contacted to an appropriate position of the user; and
the processor executes the preloaded program to acquire the ECG data, and then, the ECG data is transmitted to the portable device.

2. The device as claimed in claim 1, wherein the portable device is a mobile phone, a PDA, a MP3 player, a GPS receiver, a portable/handheld game machine, a digital camera, or a digital video camera/recorder.

3. The device as claimed in claim 1, wherein the ECG data acquisition device is wirelessly communicated with the portable device.

4. The device as claimed in claim 3, wherein the communication module is implemented to be a Bluetooth module, a wireless USB module, a WiFi module, or an IrDa module.

5. The device as claimed in claim 1, wherein the communication module is implemented to be an USB module, an UART module, an Ethernet module, or an 1394 module.

6. The device as claimed in claim 1, wherein the analog signal processing module, the analog/digital converter, and/or the processor is located outside the housing and is connected through a wire.

7. The device as claimed in claim 1, wherein the analog signal processing module, the analog/digital converter, and/or the processor are removable from the housing to connect with other electrodes for performing another ECG data acquisition.

8. The device as claimed in claim 1, wherein the ECG data is transmitted to the portable device for real-time processing and/or displaying.

9. The device as claimed in claim 1, further comprising an extension port for connecting at least a sensor/electrode.

10. The device as claimed in claim 9, wherein the sensor is an oximeter.

11. The device as claimed in claim 9, wherein the connected electrode is implemented to cooperate with the electrodes on the surface of the housing.

12. The device as claimed in claim 9, wherein the connected electrodes are implemented to replace the electrodes on the surface of the housing.

13. The device as claimed in claim 1, further comprising an oximeter for obtaining blood physiological parameters.

14. The device as claimed in claim 1, wherein the housing has a mechanical structure adjustable to adapt to different portable devices.

15. The device as claimed in claim 1, wherein the portable device provides power for the ECG data acquisition device.

16. An electrocardiographic data acquisition device for being engaged with a portable device, wherein the portable device comprises a processor to execute a preloaded program for performing an ECG data acquisition, comprising:
a housing;
at least two electrodes, located on the surface of the housing for obtaining ECG signals from a user; and
an electrical connection interface, for connecting with the portable device, wherein
the ECG data acquisition device, through the housing and the electrical connection interface thereof, is mechanically and electrically integrated with the portable device to form a combined unit;
to obtain the ECG signals, the user contacts at least one of the electrodes while holding the combined unit, and the other electrode is contacted to an appropriate position of the user; and
the processor of the portable device executes the preloaded program to acquire the ECG data, and the ECG data is processed and displayed by the portable device.

17. The device as claimed in claim 16, wherein an analog signal processing module and/or an analog/digital converter is further provided in the ECG data acquisition device and/or the portable device, for performing the ECG data acquisition.
